(19) United States
(12) Patent Application Publication
(10) Pub. No.: US 2017/0311779 A1
(43) Pub. Date: Nov. 2, 2017

(54) OPTICAL SCANNING ENDOSCOPE APPARATUS

(71) Applicant: OLYMPUS CORPORATION, Tokyo (JP)

(72) Inventor: Keiichiro NAKAJIMA, Tokyo (JP)

(73) Assignee: OLYMPUS CORPORATION, Tokyo (JP)

(21) Appl. No.: 15/597,847

(22) Filed: May 17, 2017

Related U.S. Application Data
(63) Continuation of application No. PCT/JP2014/005758, filed on Nov. 17, 2014.

(51) Int. Cl. A61B 1/00; A61B 1/06; A61B 1/04; A61B 1/07

(52) U.S. Cl. CPC A61B 1/00096; A61B 1/07; A61B 1/0669; A61B 1/00006; A61B 1/04; A61B 1/00009; A61B 1/0638

(57) ABSTRACT

This optical scanning endoscope apparatus can correct the white balance even when bending occurs in the illumination fiber. An optical scanning endoscope apparatus includes an illumination fiber that guides illumination light composed of RGB wavelengths (colors), an actuator that drives the tip of the illumination fiber and repeatedly scans the illumination light over an object, a photodetector that detects light obtained from the object by scanning of the illumination light, a signal processor that generates an image based on output of the photodetector, and a light amount detector for white balance that detects the light amount of light of RGB wavelengths from a portion of the illumination light guided by the illumination fiber. Based on the light amount of light of each of the RGB wavelengths detected by the light amount detector for white balance, the controller adjusts the white balance of the generated image.

### CROSS-REFERENCE TO RELATED APPLICATION

The present application is a Continuing Application based on International Application PCT/JP2014/005758 filed on Nov. 17, 2014, the content of which is incorporated herein by reference.

### TECHNICAL FIELD

The present disclosure relates to an optical scanning endoscope apparatus.

### BACKGROUND

A known optical scanning endoscope apparatus guides illumination light from a light source to the tip of an endoscope using a single-mode fiber (SMF), emits the illumination light towards an object, vibrates the tip of the fiber to scan the illumination light two-dimensionally over the object, and observes light such as reflected light and scattered light obtained from the object (for example, see JP 2013-121455 A (PTL 1)). In the optical scanning endoscope apparatus, from the drive waveform and drive timing for driving the tip of the optical fiber, from the detection timing of signals of received light, and the like, the pixel positions of detection signals are allocated to generate a two-dimensional image.

In order to observe a color image with the optical scanning endoscope apparatus, red (R), green (G), and blue (B) laser light sources are prepared as the light source, the optical paths of the laser light obtained from these light sources are combined, and the resulting light is scanned over an object by irradiating pulsed light of sequentially different colors. As a result, R, G, and B color components are each detected for the reflected light, scattered light, and the like obtained from the object, and after performing interpolation and the like, images in three colors are synthesized to generate a color image.

### CITATION LIST

### Patent Literature

PTL 1: JP 2013-121455 A

### SUMMARY

An optical scanning endoscope apparatus according to the present disclosure includes:

an illumination fiber configured to guide illumination light including light of a plurality of different wavelengths, the illumination fiber being supported to allow a tip thereof to oscillate;

a scanner configured to drive the tip of the illumination fiber and repeatedly scan the illumination light over an object;

a photodetector configured to detect light obtained from the object by scanning of the illumination light;

a signal processor configured to generate an image based on output of the photodetector; and

a light amount detector for white balance configured to detect a light amount of light of each of the plurality of different wavelengths from a portion of the illumination light guided by the illumination fiber;

such that based on the light amount of light of each of the plurality of different wavelengths detected by the light amount detector for white balance, the optical scanning endoscope apparatus adjusts a white balance of the image that is generated.

In one of the embodiments of the present disclosure, the optical scanning endoscope apparatus may further include a lens configured to irradiate the illumination light emitted from the illumination fiber towards the object, such that a reflector is provided at an outer peripheral portion of the lens, and the light amount detector for white balance detects at least a portion of the illumination light reflected by the reflector.

In another one of the embodiments of the present disclosure, the optical scanning endoscope apparatus may further include a lens configured to irradiate the illumination light emitted from the illumination fiber towards the object, such that the light amount detector for white balance detects at least a portion of the illumination light reflected by a surface of the lens.

In another one of the embodiments of the present disclosure, the optical scanning endoscope apparatus may further include a lens configured to irradiate the illumination light emitted from the illumination fiber towards the object, such that the light amount detector for white balance comprises a light-receiving element disposed to detect at least a portion of the illumination light at an outer peripheral portion of the lens.

In another one of the embodiments of the present disclosure, the optical scanning endoscope apparatus may further include a receiving fiber configured to receive light obtained from the object by irradiation with the illumination light and to guide the light obtained from the object to the photodetector, and a cap including a reflective area disposed to reflect at least a portion of the illumination light emitted from the illumination fiber so that the portion of the illumination light enters the receiving fiber; such that the photodetector also serves as the light amount detector for white balance.

In another one of the embodiments of the present disclosure, the optical scanning endoscope apparatus may further include a lens configured to irradiate the illumination light emitted from the illumination fiber towards the object; and

an optical demultiplexer configured to branch an optical path of reflected light from an optical path of the illumination light, the reflected light being yielded by a portion of the illumination light being reflected by a surface of the lens and propagated through the illumination fiber in light source direction;

such that the light amount detector for white balance detects the reflected light branched by the optical demultiplexer.

Another optical scanning endoscope apparatus according to the present disclosure includes:

an illumination fiber configured to guide illumination light including light of a plurality of different wavelengths, the illumination fiber being supported to allow a tip thereof to oscillate;

a probe with the illumination fiber disposed therein, including at least a flexible portion;

a scanner configured to drive the tip of the illumination fiber and repeatedly scan the illumination light over an object;

a photodetector configured to detect light obtained from the object by scanning of the illumination light;

a signal processor configured to generate an image based on output of the photodetector;

a fiber for optical loss measurement having a same bending loss characteristic as the illumination fiber and extending into the probe at least to the flexible portion; and

a light amount detector for white balance configured to detect a light amount of light of each of the plurality of different wavelengths guided by the fiber for optical loss measurement;

such that based on the light amount of light of each of the plurality of different wavelengths detected by the light amount detector for white balance, the optical scanning endoscope apparatus adjusts a white balance of the image that is generated.

The fiber for optical loss measurement may be provided to extend to the flexible portion of the probe and back.

In each of the above embodiments, based on the light amount of light of each wavelength detected by the light amount detector for white balance, the optical scanning endoscope apparatus may adjust the white balance by controlling light source to adjust an emission intensity of light of each of the plurality of different wavelengths.

Alternatively, based on the light amount of light of each wavelength detected by the light amount detector for white balance, the optical scanning endoscope apparatus may adjust the white balance by adjusting white balance of the image generated by the signal processor.

### BRIEF DESCRIPTION OF THE DRAWINGS

In the accompanying drawings:

FIG. 1 is a block diagram schematically illustrating the configuration of an optical scanning endoscope apparatus according to Embodiment 1;

FIG. 2 is a schematic overview of the scope in FIG. 1;

FIG. 3 is a cross-sectional diagram of the tip of the scope in FIG. 2;

FIG. 4 is a cross-section along the A-A line in FIG. 3;

FIG. 5 is a cross-section along the B-B line in FIG. 3;

FIG. 6A is a side view illustrating the vibration driving mechanism of the actuator and the oscillating portion of the illumination fiber in FIG. 3;

FIG. 6B is a cross-section along the A-A line in FIG. 6A;

FIG. 7 illustrates a spiral scan as an example of a scanning method;

FIG. 8 is a block diagram schematically illustrating the configuration of an optical scanning endoscope apparatus according to Embodiment 2;

FIG. 9 is a cross-sectional diagram of the tip of the scope in FIG. 8;

FIG. 10 is a cross-sectional diagram illustrating the tip of the scope in an optical scanning endoscope apparatus according to Embodiment 3;

FIG. 11 is a cross-section along the A-A line in FIG. 10;

FIG. 12 is a block diagram schematically illustrating the configuration of an optical scanning endoscope apparatus according to Embodiment 4;

FIG. 13 is a cross-sectional diagram of the tip of the scope in FIG. 12;

FIG. 14 is a view of the cap in FIG. 13 seen along the optical axis of the projection lenses;

FIG. 15 is a block diagram schematically illustrating the configuration of an optical scanning endoscope apparatus according to Embodiment 5;

FIG. 16 illustrates the arrangement of the fiber for optical loss measurement in FIG. 15; and

FIG. 17 is a block diagram schematically illustrating the configuration of an optical scanning endoscope apparatus according to Embodiment 6.

### DETAILED DESCRIPTION

Embodiments of the present disclosure are described below with reference to the drawings.

### Embodiment 1

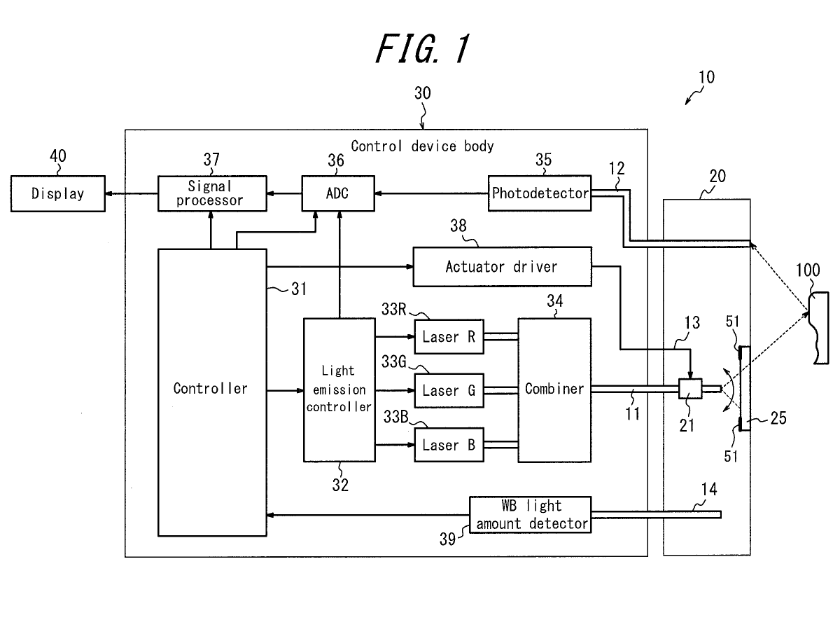

With reference to FIGS. 1 to 7, Embodiment 1 is described. FIG. 1 is a block diagram schematically illustrating the configuration of an optical scanning endoscope apparatus according to Embodiment 1. In FIG. 1, an optical scanning endoscope apparatus 10 includes a scope 20, a control device body 30, and a display 40.

First, the configuration of the control device body 30 is described. The control device body 30 includes a controller 31 that controls the optical scanning endoscope apparatus 10 overall, a light emission controller 32, lasers 33R, 33G, and 33B (the lasers 33R, 33G, and 33B also being collectively referred to below as a “light source 33”), a combiner 34, a photodetector 35 (photodetector), an analog/digital converter (ADC) 36, a signal processor 37, an actuator driver 38, and a light amount detector for white balance (WB light amount detector) 39.

In accordance with control by the light emission controller 32, the light source 33 constituted by the lasers 33R, 33G, and 33B selectively emits light of a plurality of different wavelengths (in this embodiment, light of three wavelengths: R, G, and B). As used herein, “selectively emits light of a plurality of different wavelengths” refers to light of one wavelength selected by the light emission controller 32 being emitted at a timing selected by the light emission controller 32. For example, Diode-Pumped Solid-State (DPSS) lasers or laser diodes may be used as the lasers 33R, 33G, and 33B.

In response to a control signal from the controller 31, the light emission controller 32 controls the light emission timing of the light source 33. In this embodiment, during one scan, the light emission controller 32 switches the wavelength of the R, G, or B light from the light source 33 in a predetermined light emission order (for example, in the order R, G, B) at constant time intervals. As used here, “one scan” refers to one scan, in order to capture one image, from the starting point to the ending point of a predetermined scan path, such as a spiral.

The laser light emitted from the lasers 33R, 33G, and 33B passes through optical paths joined coaxially by the combiner 34 and is incident as illumination light on an illumination fiber 11, which is a single-mode fiber (SMF). The combiner 34 may, for example, be configured using a fiber multiplexer, a dichroic prism, or the like. The lasers 33R, 33G, and 33B and the combiner 34 may be stored in a housing that is separate from the control device body 30 and is joined to the control device body 30 by a signal wire.

Light incident on the illumination fiber 11 from the combiner 34 is guided to the tip of the scope 20 and irradiated onto an object 100. At this time, by driving the actuator 21 of the scope 20 by vibration, the actuator driver 38 of the control device body 30 drives the tip of the illumination fiber 11 by vibration. Accordingly, the scanner is configured to include the actuator driver 38 and the actuator 21. As a result, the illumination light emitted from the illumination fiber 11 scans the observation surface of the object 100 in 2D repeatedly over a predetermined scan path. Light such as reflected light or scattered light that is obtained from the object 100 by irradiation with the illumination light is received at the tip of a receiving fiber 12, which is constituted by a multi-mode fiber (MMF), and is guided through the scope 20 to the control device body 30.

The photodetector 35 detects light from the object 100 through the receiving fiber 12, the light being obtained by irradiation of light at the wavelength (also referred to below as the color) of one of R, G, and B in each light emission cycle of the light source 33, and outputs an analog signal (electrical signal).

The ADC 36 converts the analog signal output from the photodetector 35 to a digital signal (electrical signal) and outputs the result to the signal processor 37.

The signal processor 37 associates the digital signals, which correspond to the various wavelengths and were input from the ADC 36, with the respective light emission timings and scanning positions, and stores the results sequentially in memory (not illustrated). Information on the light emission timing and scanning position is acquired from the controller 31. The controller 31 calculates information on the scanning position along the scan path from information such as the amplitude and phase of vibration voltage applied by the actuator driver 38. After completion of scanning or during scanning, the signal processor 37 generates an image signal while performing image processing as necessary, such as enhancement, γ processing, and interpolation, based on each digital signal input from the ADC 36 and displays an image of the object 100 on the display 40.

A light amount balance detection fiber 14 is a multi-mode fiber (MMF) that extends from the control device body 30 to near the tip of the scope 20. A portion of the illumination light emitted from the illumination fiber 11 enters the edge of the light amount balance detection fiber 14 at the scope 20 side and is guided to the light amount detector for white balance 39. During one scan, the amount of illumination light received by the light amount balance detection fiber 14 is configured to be a constant ratio of the total amount of illumination light emitted from the illumination fiber 11.

The light amount detector for white balance 39 detects the light amount of light at each of the different wavelengths R, G, and B from the portion of illumination light that is guided through the light amount balance detection fiber 14. In this embodiment, the lasers 33R, 33G, and 33B emit light at successively selected timings. Therefore, the light amount detector for white balance 39 can detect the light of each wavelength in synchronization with the emission timing of light of each wavelength. The controller 31 is notified of the detected light amount at each wavelength.

Based on the light amount of light of each of the wavelengths of R, G, and B detected by the light amount detector for white balance 39, the controller 31 calculates the correction amount for each of the wavelengths of R, G, and B. The correction amount in this case may, for example, be provided as a scale factor of the illumination light intensity of R, G, and B necessary to adjust the white balance. The controller 31 can control the light emission controller 32 to change the emission intensity of the lasers 33R, 33G, and 33B.

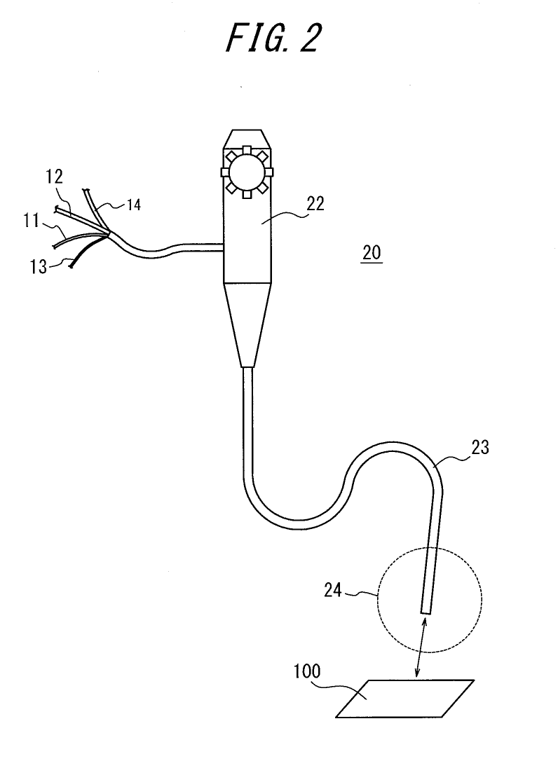

Next, the configuration of the scope 20 is described. FIG. 2 is a schematic overview of the scope 20. The scope 20 includes an operation part 22 and an insertion part 23 (probe). The illumination fiber 11, receiving fiber 12, wiring cables 13, and light amount balance detection fiber 14 from the control device body 30 are each connected to the operation part 22. The illumination fiber 11, receiving fiber 12, wiring cables 13, and light amount balance detection fiber 14 pass through the insertion part 23 and extend to a tip 24 (the portion within the dotted line in FIG. 2) of the insertion part 23.

The insertion part 23 is flexible, except for the hard tip 24. In particular, the portion near the tip 24 is configured to be bendable, and the tip 24 can be pointed in any direction.

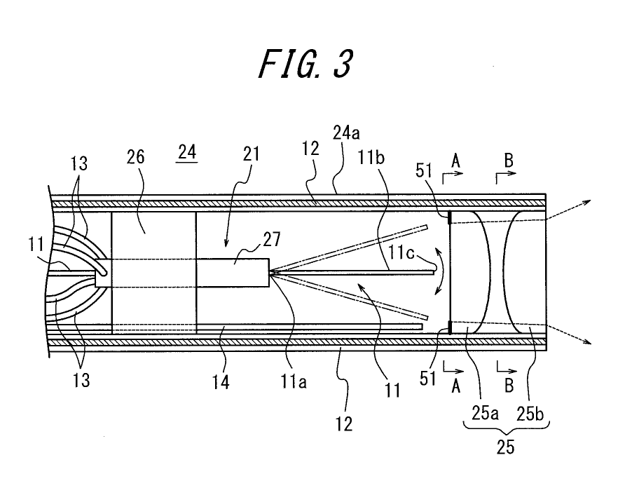

FIG. 3 is a cross-sectional diagram illustrating an enlargement of the tip 24 of the insertion part 23 of the scope 20 in FIG. 2. The outer periphery of the tip 24 of the scope 20 is covered by a hard, cylindrical outer tube 24a. The tip 24 includes the actuator 21, projection lenses 25 (inner lens 25a, outer lens 25b), the illumination fiber 11, which passes through the central portion of the tip 24, a plurality of receiving fibers 12 that pass through the outer tube 24a, and the light amount balance detection fiber 14, which is provided along the inner periphery of the outer tube 24a.

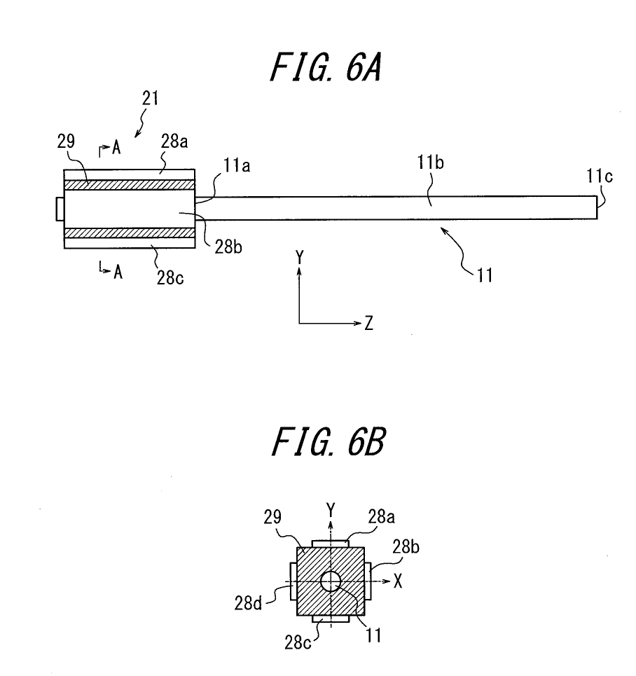

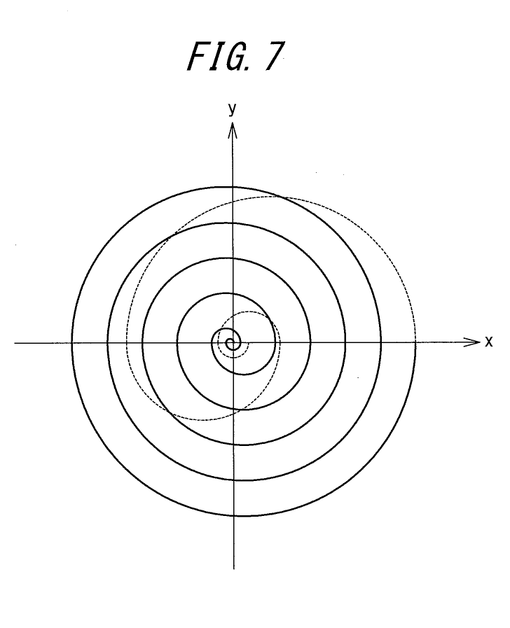

The actuator 21 is a member that drives a tip 11c of the illumination fiber 11 by vibration. The actuator 21 includes an actuator tube 27 fixed by an attachment ring 26 fixed to the inside of the outer tube 24a, a flexible fiber holding member 29 disposed inside the actuator tube 27, and piezoelectric elements 28a to 28d (see FIGS. 6A and 6B). The illumination fiber 11 is supported by the fiber holding member 29, and the portion from a fixed end 11a supported by the fiber holding member 29 to the tip 11c is an oscillating part 11b that is supported to allow oscillation. The receiving fibers 12 are disposed to pass through the outer tube 24a and extend to the end of the tip 24.

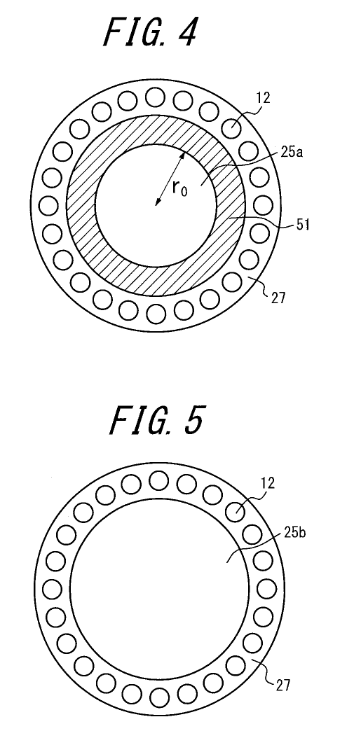

Furthermore, the projection lenses 25 are configured by two convex lenses, i.e. an inner lens 25a and an outer lens 25b, and are disposed at the extreme end of the tip 24 of the insertion part 23 in the scope 20. The projection lenses 25 are configured so that laser light emitted from the tip 11c of the illumination fiber 11 is irradiated on the object 100 and roughly concentrated. Of the two projection lenses 25, the inner lens 25a positioned on the illumination fiber 11 side is a plano-convex lens with the convexity facing the object 100. As illustrated in FIG. 3 and FIG. 4, which is a cross-section along the A-A line in FIG. 3, a reflector 51 is provided in the outer peripheral portion of the plane on the illumination fiber 11 side of the inner lens 25a. In order to reflect illumination light from the illumination fiber 11, the reflector 51 is configured for example by vapor deposition of silver, aluminum, or the like on the planar portion of the inner lens 25a. Of the two projection lenses 25, the outer lens 25b disposed on the object 100 side is a plano-convex lens with the convexity facing the illumination fiber 11. As illustrated in FIG. 3 and FIG. 5, which is a cross-section along the B-B line in FIG. 3, no reflector is provided on the outer lens 25b, unlike the inner lens 25a. The projection lenses 25 are not limited to a double lens configuration and may be configured as a single lens or as three or more lenses. Also, the projection lenses 25 may be configured by lenses of a variety of shapes.

The light amount balance detection fiber 14 extends along the inner periphery of the outer tube 24a to near the inner lens 25a. As a result, the light amount balance detection fiber 14 can receive light that is measurement light from the illumination fiber 11 reflected by the reflector 51 of the inner lens 25a. In FIGS. 1 to 3, the light amount balance detection fiber 14 is displayed as one fiber, but a plurality of fibers may be used.

FIG. 6A illustrates the vibration driving mechanism of the actuator 21 of the optical scanning endoscope apparatus 10 and illustrates the oscillating part 11b of the illumination fiber 11. FIG. 6B is a cross-sectional diagram along the A-A line in FIG. 6A. The vibration driving mechanism includes the piezoelectric elements 28a to 28d and the fiber holding member 29. The illumination fiber 11 passes through the center of the fiber holding member 29, which is shaped as a quadratic prism, and is fixed and held by the fiber holding member 29. The four sides of the fiber holding member 29 respectively face the ±Y direction and the ±X direction. A pair of piezoelectric elements 28a and 28c for driving in the Y direction are fixed onto the sides of the fiber holding member 29 in the ±Y direction, and a pair of piezoelectric elements 28b and 28d for driving in the X direction are fixed onto the sides in the ±X direction.

The wiring cable 13 from the actuator driver 38 of the control device body 30 is connected to the piezoelectric elements 28a to 28d, which are driven by application of voltage by the actuator driver 38.

The pair of piezoelectric elements 28b and 28d in the X direction may, for example, be piezoelectric elements with the same direction of expansion and contraction relative to the application direction of voltage, and voltage of equivalent magnitude and opposite sign may always be applied. One of the piezoelectric elements 28b and 28d disposed opposite each other with the fiber holding member 29 therebetween expands and the other contracts, thereby causing the fiber holding member 29 to flex. Repeating this operation produces vibration in the X direction. The same is true for vibration in the Y direction as well.

The actuator driver 38 can perform vibration driving of the piezoelectric elements 28b and 28d for driving in the X direction and the piezoelectric elements 28a and 28c for driving in the Y direction by applying vibration voltage of the same frequency or vibration voltage of different frequencies thereto. Upon vibration driving of the piezoelectric elements 28a and 28c for driving in the Y direction and the piezoelectric elements 28b and 28d for driving in the X direction, the oscillating part 11b of the illumination fiber 11 illustrated in FIGS. 3 and 6A vibrates, and the tip 11c is deflected, so that the laser light emitted from the tip 11c sequentially scans the surface of the object 100 over a predetermined scan path.

In this embodiment, with the aforementioned vibration driving mechanism, the object 100 is scanned over a spiral scan path. During each scan, a vibration voltage for vibration in a predetermined cycle starting from an amplitude of 0 while expanding to a predetermined maximum is applied to the piezoelectric elements 28b and 28d for driving in the X direction. At the same time that the vibration voltage is applied to the piezoelectric elements 28b and 28d for driving in the X direction, vibration voltage with the same cycle and amplitude as the vibration voltage for driving the piezoelectric elements 28b and 28d but shifted 90° in phase is applied to the piezoelectric elements 28a and 28c for driving in the Y direction. When the amplitude reaches its maximum, application of voltage to the piezoelectric elements 28a to 28d is suspended, or voltage that is controlled so as to reduce the amplitude is applied, and the amplitude of the tip 11c of the illumination fiber 11 diminishes rapidly. In this way, the illumination fiber 11 repeatedly scans over a spiral scan path.

The controller 31 controls light emission of the lasers 33R, 33G, and 33B via the light emission controller 32 in synchronization with the driving of the tip 11c of the illumination fiber 11 by the actuator driver 38. The lasers 33R, 33G, and 33B are controlled to emit light sequentially as the amplitude is increasing, and after the amplitude reaches its maximum, to suspend light emission while the amplitude diminishes. In this way, the tip 11c of the illumination fiber 11 is driven over a path such as the one indicated by the solid line in FIG. 7 to scan over the object 100 in a spiral scan path. The dashed line in FIG. 7 indicates the path of the tip 11c as the amplitude diminishes. FIG. 7 is only a conceptual diagram of a scan, and an actual scan is denser in the radial direction.

According to the above configuration, the optical scanning endoscope apparatus 10 observes the object 100 and adjusts the white balance as described below.

The controller 31 controls the light source 33 via the light emission controller 32 and sequentially emits light of R, G, and B wavelengths. The optical paths of the emitted light of R, G, and B wavelengths are combined by the combiner 34 and are guided to the scope 20 by the illumination fiber 11. Simultaneously, via the actuator driver 38, the controller 31 causes the actuator 21 to drive the oscillating part 11b of the illumination fiber 11 in a spiral scan. When the scanning amplitude of the illumination light emitted from the tip 11c of the illumination fiber 11 (the radius from the center of the spiral scan) at the position where the illumination light passes through the inner lens 25a is smaller than the radius of the reflector 51 at the inner periphery thereof, the illumination light passes through the inner lens 25a and the outer lens 25b and irradiates the object 100. As a result of this irradiation, the reflected light, scattered light, and the like obtained from the object 100 are received by the receiving fibers 12, detected by the photodetector 35, converted to a digital signal by the ADC 36, and stored as pixel information associated by the signal processor 37 with coordinate information of the object.

The signal processor 37 acquires one frame worth of pixel information while the illumination light expands in amplitude from the scan center over a range passing through the inner portion of the reflector 51 of the inner lens 25a. Accordingly, the reflector 51 of the inner lens 25a is disposed along the optical path of illumination light that is not used for image generation.

Furthermore, upon expanding the scanning amplitude of the illumination fiber 11 so that the scanning amplitude of the illumination light at a position passing through the inner lens 25a (the radius from the center of the spiral scan) becomes greater than the radius of the reflector 51 at the inner periphery thereof (r₀ in FIG. 4), the illumination light is reflected by the reflector 51. A portion of the reflected illumination light enters the light amount balance detection fiber 14 and is detected by the light amount detector for white balance 39. The light amount detector for white balance 39 outputs the detected light amount to the controller 31. Based on the timing at which the controller 31 instructed the light emission controller 32 to emit light, the controller 31 can identify whether the reflected light that was received is R, G, or B wavelength length. For example at each scan, the controller 31 calculates the total light amount of R, G, and B wavelength reflected light detected by the light amount detector for white balance 39 and monitors the change in the total light amount.

In this way, the optical scanning endoscope apparatus 10 is operated by the operation part 22 while reflected light from the illumination light reflected by the reflector 51 is being monitored. When the insertion part 23 is bent, bending loss occurs in the illumination light propagated by the illumination fiber 11. Since the illumination fiber 11 is a single-mode fiber (SMF), the loss ratio due to bending differs by wavelength. Based on the change in the light amount of R, G, and B wavelength reflected light, the controller 31 calculates the correction amounts for light of different wavelengths in order to correct the white balance. For example, the light amounts of R, G, and B wavelength reflected light when the white balance is good, with no loss due to bending in the insertion part 23, are measured in advance, and each light amount is set to one. If the insertion part 23 is bent and the light amounts of R, G, and B wavelength reflected light detected by the light amount detector for white balance 39 are respectively reduced by the ratios Rᵣ, R_{g}, and R_{b} (Rᵣ, R_{g}, R_{b}<1), then the white balance can be corrected by adjusting the emission intensity of R, G, and B wavelengths of illumination light so as to be scaled respectively by 1/Rᵣ, 1/R_{g}, and 1/R_{b}. Accordingly, the controller 31 calculates the correction amounts as 1/Rᵣ, 1/R_{g}, and 1/R_{b}.

Next, the controller 31 transmits the correction amounts to the light emission controller 32 and accordingly changes the emission intensity of the lasers 33R, 33G, and 33B. As a result, even when loss that differs depending on wavelength is produced by bending of the illumination fiber 11, the balance of R, G, and B wavelengths of light can be maintained in the illumination light emitted from the tip 11c of the illumination fiber 11.

Since the receiving fibers 12 and the light amount balance detection fiber 14 are multi-mode fibers (MMF), the bending loss does not depend as strongly on wavelength as for a single-mode fiber (SMF). Therefore, the white balance can be adjusted by correcting only the difference in the loss ratio for the illumination fiber 11 that is a single-mode fiber (SMF).

As described above, according to this embodiment, the illumination light propagated through the illumination fiber 11 is reflected, and the light amount detector for white balance 39 that detects light at each of the wavelengths of R, G, and B from a portion of the reflected light is provided.

Based on the detected light amount of light of each of the wavelengths of R, G, and B, correction amounts for correcting the emission intensity of the lasers 33R, 33G, and 33B are calculated, and the white balance of the generated image is adjusted. Therefore, even when the illumination fiber is bent, the white balance can be adjusted.

(Modification)

In Embodiment 1, based on the correction amounts from the controller, the emission intensity of the light source is changed. The white balance may also be adjusted, however, by correcting the intensity of pixel signals in the signal processor 37. In this case, the controller 31 transmits the correction amounts for the signals of each of the colors R, G, and B to the signal processor 37, and the signal processor 37 multiplies each of the R, G, and B pixels by the corresponding correction amount. The white noise of the resulting image can be adjusted in this case without changing the light amount of the lasers 33R, 33G, and 33B in the light source 33.

### Embodiment 2

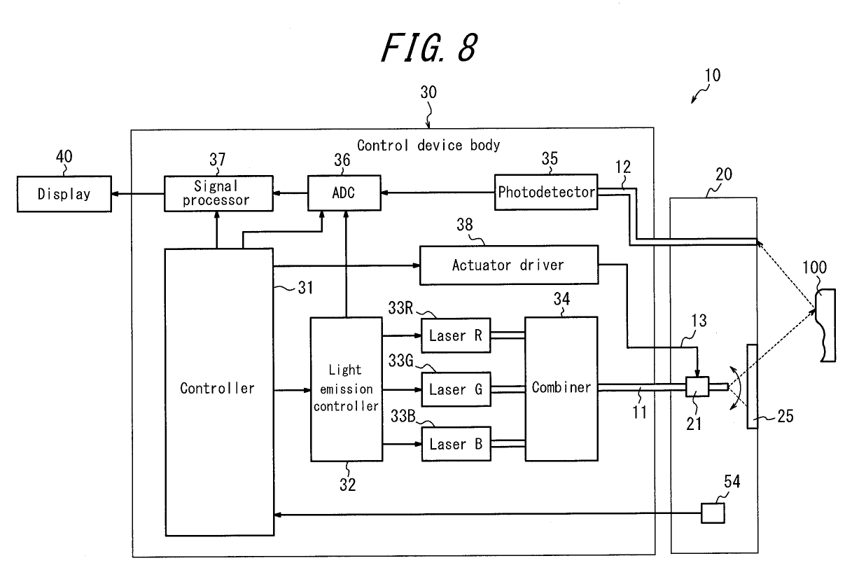

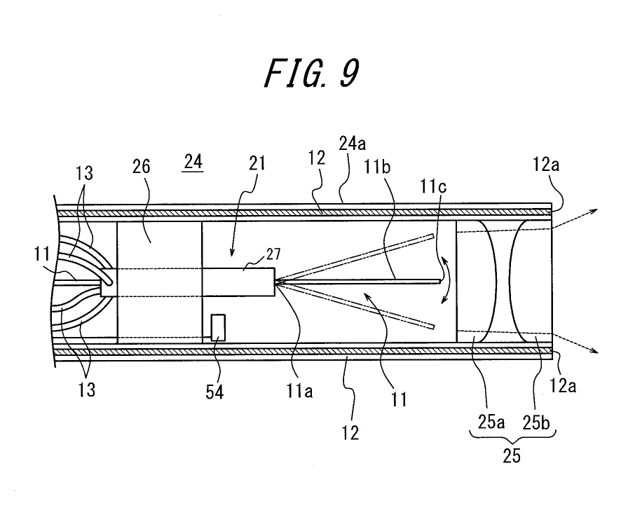

FIG. 8 is a block diagram schematically illustrating the configuration of an optical scanning endoscope apparatus according to Embodiment 2 of the present disclosure. FIG. 9 is a cross-sectional diagram of the tip of the scope in FIG. 8. This embodiment differs from the optical scanning endoscope apparatus 10 according to Embodiment 1 by not providing the reflector 51 on the projection lenses 25, but rather detecting reflected light that is reflected by Fresnel reflection of the inner lens 25a. In order to detect reflected light, a reflected light receiver 54 disposed in the tip 24 of the scope 20 is used.

Therefore, neither the light amount balance detection fiber 14 nor the light amount detector for white balance 39 is provided. The reflected light receiver 54 is an element that converts an optical signal into an electrical signal and is disposed so that the light receiving surface faces a plane of the inner lens 25a. A photodiode (PD), for example, may be used as the reflected light receiver 54.

The reflected light receiver 54 is connected to the controller 31 by a wiring line that passes through the scope 20. Since the remaining configuration and effects are similar to those of Embodiment 1, identical or corresponding constituent elements are labeled with the same reference signs, and a description thereof is omitted.

According to this embodiment, Fresnel reflection of the inner lens 25a is detected, without providing the reflector 51. Therefore, in addition to the effects of Embodiment 1, the balance of the R, G, and B light amounts of the illumination light can always be monitored without regard to the scan position over the object 100. Furthermore, there is no need to pass the light amount balance detection fiber 14 through the insertion part 23 of the scope 20. Therefore, the insertion part 23 can be configured to be thinner.

### Embodiment 3

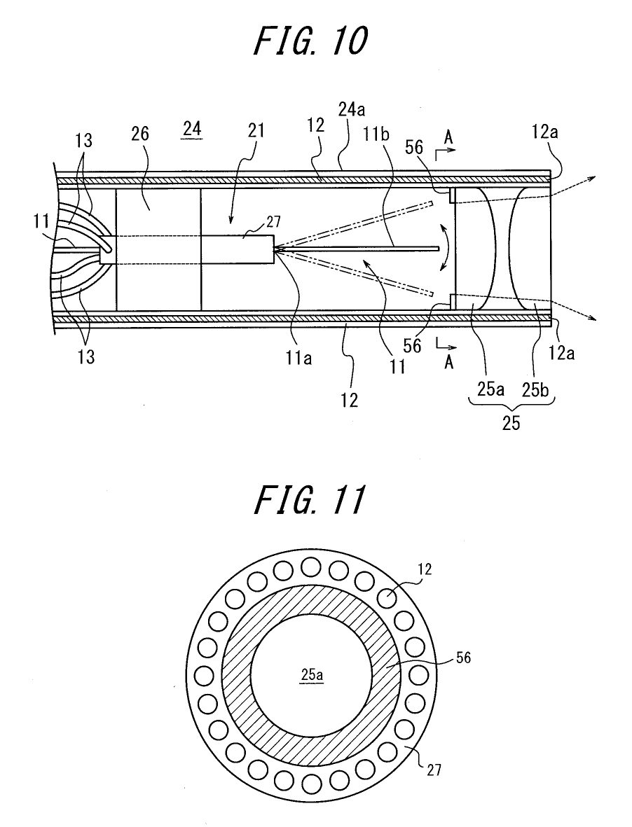

FIG. 10 is a block diagram schematically illustrating the configuration of an optical scanning endoscope apparatus according to Embodiment 3 of the present disclosure. FIG. 11 is a cross-section along the A-A line in FIG. 10. This embodiment differs from the optical scanning endoscope apparatus 10 according to Embodiment 2 in that, instead of the reflected light receiver 54, a reflected light receiver 56 (light-receiving element) is disposed along the outer periphery of the incident plane of the inner lens 25a. The reflected light receiver 56 is connected electrically to the controller 31 by non-illustrated wiring. Since the remaining configuration and effects are similar to those of Embodiment 2, identical or corresponding constituent elements are labeled with the same reference signs, and a description thereof is omitted.

According to this embodiment, as in Embodiment 2, there is no need to pass the light amount balance detection fiber 14 through the insertion part 23 of the scope 20. Therefore, in addition to the effects of Embodiment 1, the insertion part 23 can be configured to be thinner.

### Embodiment 4

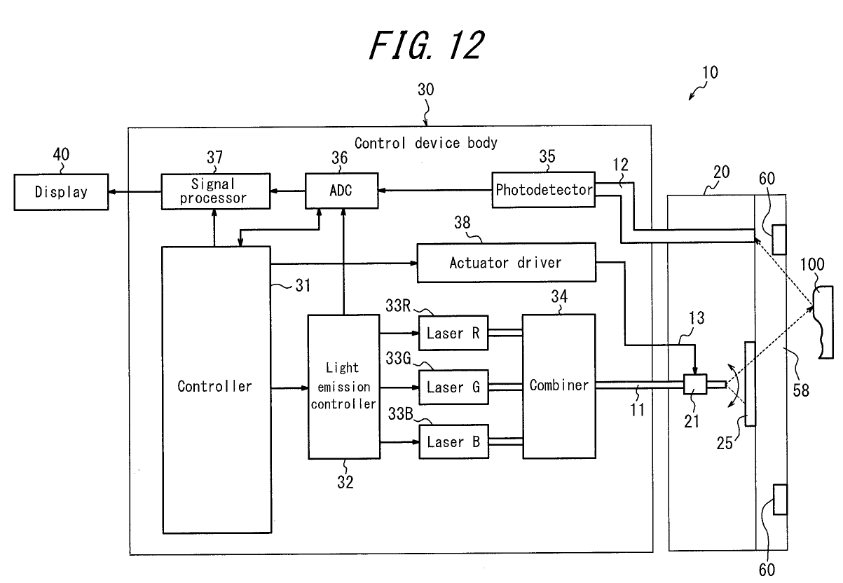

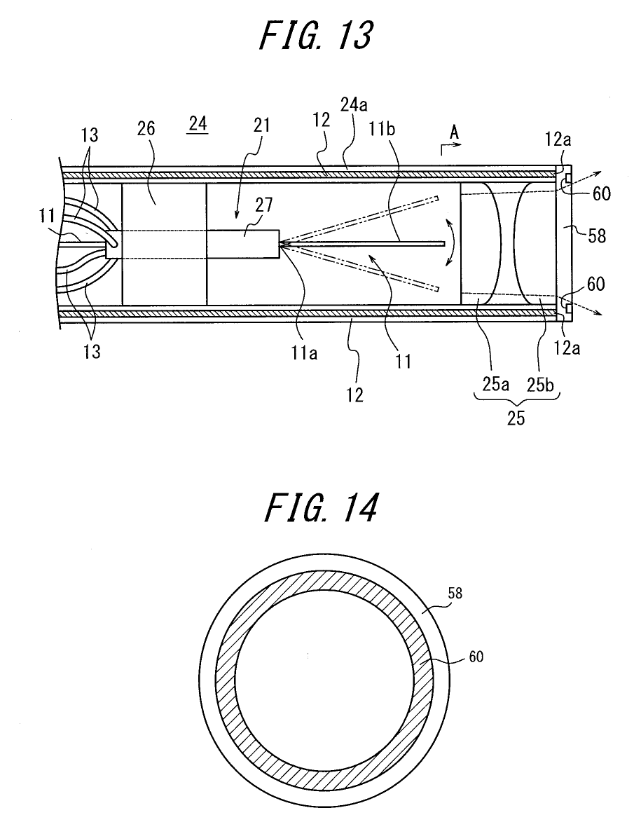

FIG. 12 is a block diagram schematically illustrating the configuration of an optical scanning endoscope apparatus according to Embodiment 4 of the present disclosure. FIG. 13 is a cross-sectional diagram of the tip of the scope in FIG. 12. The optical scanning endoscope apparatus 10 according to this embodiment differs from the optical scanning endoscope apparatus 10 according to Embodiment 1 in that, instead of the reflector 51 of the inner lens 25a, a cap 58 is mounted on the tip of the scope so as to cover the tip. The cap 58 is formed by a disk-shaped, transparent material (such as glass). As illustrated by FIG. 14, an annular reflective area 60 is provided inside the cap 58, along the outer periphery of the cap 58. The face of the cap 58 on which the reflective area 60 is formed is separated from the entrance surface of the receiving fibers 12. The reflective area 60 is formed on the outside of the optical path of illumination light used for image formation and is disposed so that, when the scanning amplitude of illumination light expands and the illumination light is reflected by the reflective area 60 during one scan, at least a portion of the reflected light enters the receiving fibers 12. On the other hand, neither the light amount balance detection fiber 14 nor the light amount detector for white balance 39 of Embodiment 1 is provided. Since the remaining configuration and effects are similar to those of Embodiment 1, identical or corresponding constituent elements are labeled with the same reference signs, and a description thereof is omitted.

According to the above configuration, in this embodiment, when the illumination fiber 11 is driven and illumination light passes through the inside of the reflective area 60 of the cap 58 to scan the object 100 during one scan, then as in Embodiment 1, reflected light and scattered light from the object 100 is received by the receiving fibers 12, and from the signal detected by the photodetector 35, pixel data is stored and an image is formed in the signal processor 37. Furthermore, when the illumination light with an expanded scanning amplitude is reflected by the reflective area 60, the reflected light is received by the receiving fibers 12 and detected by the photodetector 35. The light amount of reflected light received by the photodetector 35 is input into the controller 31 via the ADC 36. Based on the light emission timing of the light emission controller 32, the controller 31 identifies whether the signal of reflected light is R, G, or B wavelength light and calculates the total light amount of light of each of the R, G, and B wavelengths in each scan. Accordingly, in this embodiment, the photodetector 35 also functions as the light amount detector for white balance 39 in Embodiment 1. As a result, based on the light amount of detected light of each of the R, G, and B wavelengths, the controller 31 adjusts the white balance of the generated image in a similar way as in Embodiment 1.

According to this embodiment, there is no need to pass the light amount balance detection fiber 14 through the insertion part 23 of the scope 20. Therefore, in addition to the effects of Embodiment 1, the insertion part 23 can be configured to be thinner. Also, since the light amount detector for white balance 39 is not provided, the configuration of the optical scanning endoscope apparatus 10 is simplified.

### Embodiment 5

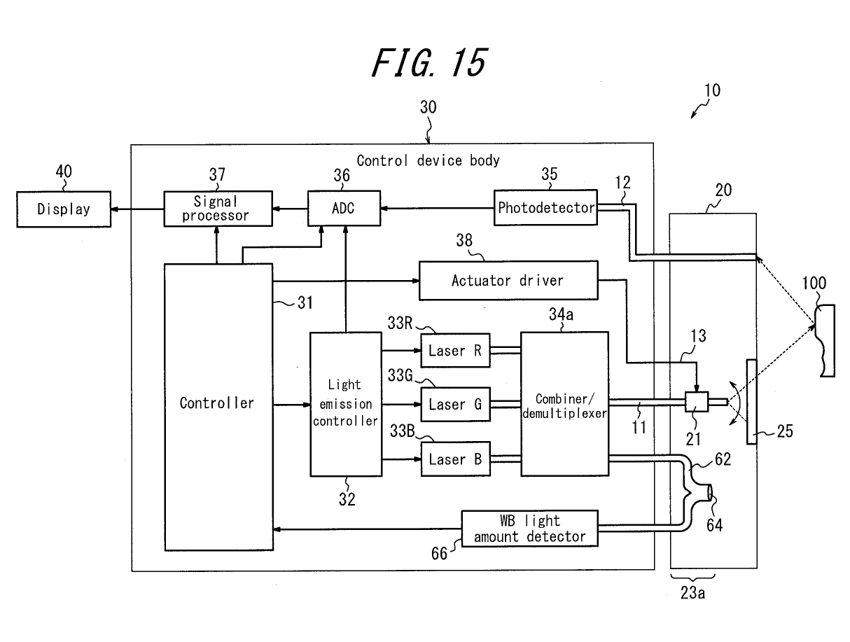

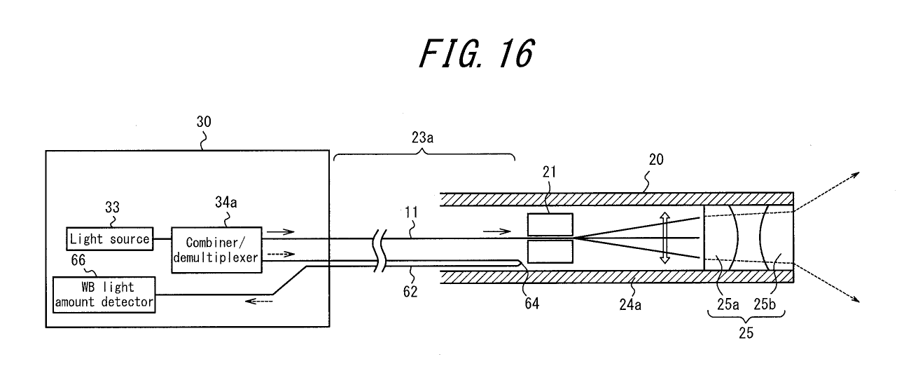

FIG. 15 is a block diagram schematically illustrating the configuration of an optical scanning endoscope apparatus according to Embodiment 5 of the present disclosure. FIG. 16 illustrates the arrangement of the fiber for optical loss measurement in FIG. 15. For the sake of simplicity, FIG. 16 partially omits or simplifies the configuration of each component. This embodiment differs from Embodiment 1 by providing a combiner/demultiplexer 34a instead of the combiner 34. Also, two fibers for optical loss measurement 62 are provided instead of the light amount balance detection fiber 14. The combiner/demultiplexer 34a coaxially combines the optical paths of the lasers 33R, 33G, and 33B while also branching a portion of output to one of the fibers for optical loss measurement 62. The fibers for optical loss measurement 62 have the same bending loss characteristic as the illumination fiber 11 and are single-mode fibers (SMF) that extend into the scope 20 at least to a flexible portion 23a of the insertion part 23 and back. The two fibers for optical loss measurement 62 are fused near the tip 24 of the scope 20, and the fused end face is a reflecting surface 64. The other fiber for optical loss measurement 62 that is not connected to the combiner/demultiplexer is connected to the light amount detector for white balance 66. In this embodiment, no reflector 51 is provided in the inner lens 25a, unlike Embodiment 1. Since the remaining configuration and effects are similar to those of Embodiment 1, identical or corresponding constituent elements are labeled with the same reference signs, and a description thereof is omitted.

According to the above configuration, when the optical scanning endoscope apparatus 10 of this embodiment observes the object 100, a portion of the illumination light emitted from the light source 33 is branched by the combiner/demultiplexer 34a, travels down and back over the two fibers for optical loss measurement 62 by being reflected back by the reflecting surface 64, and is detected by the light amount detector for white balance 66. The fibers for optical loss measurement 62 have the same bending loss characteristic as the illumination fiber 11. Therefore, based on the light amount of each of the R, G, and B wavelengths output from the light amount detector for white balance 66, the controller 31 can monitor the change in the amount of loss produced by bending the illumination fiber 11. As a result, the controller 31 can adjust the white balance of the generated image in a similar way as in Embodiment 1.

Accordingly, with this embodiment, in addition to achieving the effects of Embodiment 1, it is not necessary to provide the reflector 51 on the projection lenses 25. Furthermore, this embodiment offers the advantage of not being affected by external light entering through the projection lenses 25. Also, the change in the white balance can be monitored constantly regardless of the scanning amplitude of the illumination fiber 11.

### Embodiment 6

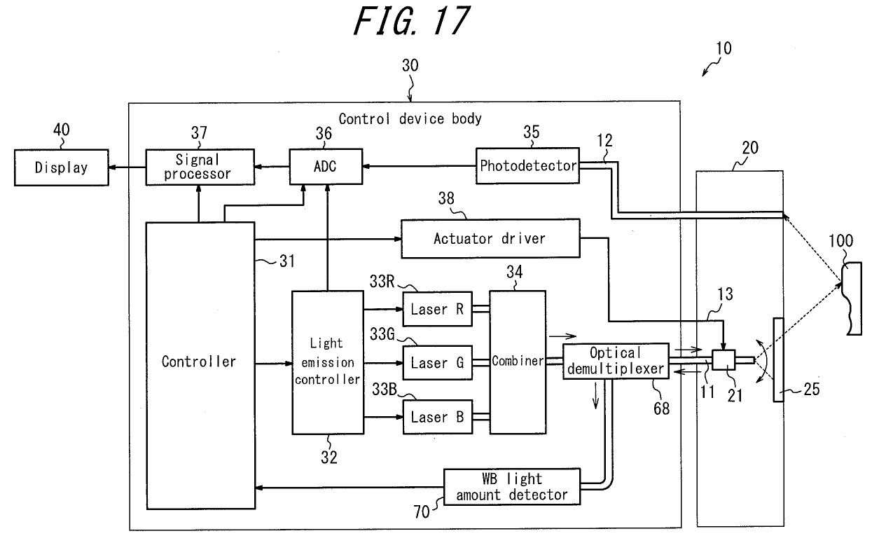

FIG. 17 is a block diagram schematically illustrating the configuration of an optical scanning endoscope apparatus according to Embodiment 6. In Embodiment 6, a portion of the illumination light that passes through the illumination fiber 11 and is emitted from the tip is reflected by the projection lens 25, and the reflected light is received by the same illumination fiber 11, guided to the control device body 30, and detected. Therefore, in the optical scanning endoscope apparatus 10 of Embodiment 1, an optical demultiplexer 68 is provided between the combiner 34 and the illumination fiber 11, and a light amount detector for white balance 70 is connected to one output side of the optical demultiplexer 68. The optical demultiplexer 68 emits illumination light from the combiner 34 to the illumination fiber 11 and also branches reflected light from the optical path of the illumination light and emits the branched reflected light to the light amount detector for white balance 70, the reflected light being reflected by a surface of the projection lens 25 and propagated through the illumination fiber 11 in the light source 33 direction (for the sake of explanation, in FIG. 17, the directions in which light proceeds around the optical demultiplexer 68 are indicated by arrows). Accordingly, the illumination fiber 11 of this embodiment also serves as the light amount balance detection fiber 14 of Embodiment 1. In this embodiment, no reflector 51 is provided in the inner lens 25a, unlike Embodiment 1. Since the remaining configuration and effects are similar to those of Embodiment 1, identical or corresponding constituent elements are labeled with the same reference signs, and a description thereof is omitted.

According to the above configuration, when the optical scanning endoscope apparatus 10 of this embodiment observes the object 100, a portion of the illumination light emitted from the illumination fiber 11 at the tip of the scope 20 is reflected by the projection lens 25, reenters the illumination fiber 11, and is propagated to the optical demultiplexer 68 as returning light. This returning light is output from the optical demultiplexer 68 to the light amount detector for white balance 70 and is detected. Based on the light amount of each of the R, G, and B wavelengths output from the light amount detector for white balance 70, the controller 31 can monitor the change in the amount of loss produced by bending the illumination fiber 11. As a result, the controller 31 can adjust the white balance of the generated image in a similar way as in Embodiment 1.

Accordingly, with this embodiment, in addition to achieving the effects of Embodiment 1, it is not necessary to provide the reflector 51 on the projection lens 25. There is also no need to pass the light amount balance detection fiber 14 through the insertion part 23 of the scope 20. Therefore, the insertion part 23 can be configured to be thinner. Furthermore, there is no need to provide a light-receiving element in the scope 20, thus offering the advantage of simplifying the configuration of the scope 20.

The present disclosure is not limited to the above embodiments, and a variety of changes and modifications may be made. For example, the driving mechanism for scanning the illumination fiber is not limited to piezoelectric elements. An electromagnetic force, for example, may be used. The method for scanning the illumination fiber is not limited to a spiral scan. Scanning may be performed by a raster scan, Lissajous scan, or other scanning mode. For example, in the case of Embodiment 1, when adopting a raster scan or a Lissajous scan, the reflector may be a rectangular frame that surrounds a rectangular region used for image generation. The light source has been described as sequentially emitting light of R, G, and B wavelengths, but this example is not limiting. Lasers that emit light of other wavelengths may be used as the light source, or a combination of four or more light sources may be used. Also, lasers that emit R, G, and B laser light may be caused to produce pulsed emissions at the same timing, and the emissions may be multiplexed by a combiner to yield white light that is irradiated on an object. In this case, in the photodetector and the light amount detector for white balance, separating means using a dichroic mirror or the like for separating the light into each wavelength component becomes necessary.

### REFERENCE SIGNS LIST

10 Optical scanning endoscope apparatus

11 Illumination fiber

11a Fixed end

11b Oscillating part

11c Tip

12 Receiving fiber

13 Wiring cable

14 Light amount balance detection fiber

20 Scope

21 Actuator

22 Operation part

23 Insertion part (probe)

23a Flexible portion

24 Tip

24a Outer tube

25 Projection lens

25a Inner lens

25b Outer lens

26 Attachment ring

27 Actuator tube

28a to 28d Piezoelectric element

29 Fiber holding member

30 Control device body

31 Controller

32 Light emission controller

33 Light source

33R, 33G, 33B Laser

34 Combiner

34a Combiner/demultiplexer

35 Photodetector

36 ADC

37 Signal processor

38 Actuator driver

39 Light amount detector for white balance (WB light amount detector)

40 Display

51 Reflecting portion

54 Reflected light receiver

56 Reflected light receiver

58 Cap

60 Reflective area

62 Fiber for optical loss measurement

64 Reflecting surface

66 Light amount detector for white balance (WB light amount detector)

68 Optical demultiplexer

70 Light amount detector for white balance (WB light amount detector)

100 Object

1. An optical scanning endoscope apparatus comprising:
an illumination fiber configured to guide illumination light including light of a plurality of different wavelengths, the illumination fiber being supported to allow a tip thereof to oscillate;
a scanner configured to drive the tip of the illumination fiber and repeatedly scan the illumination light over an object;
a photodetector configured to detect light obtained from the object by scanning of the illumination light;
a signal processor configured to generate an image based on output of the photodetector; and
a light amount detector for white balance configured to detect a light amount of light of each of the plurality of different wavelengths from a portion of the illumination light guided by the illumination fiber;
wherein based on the light amount of light of each of the plurality of different wavelengths detected by the light amount detector for white balance, the optical scanning endoscope apparatus adjusts a white balance of the image that is generated.

2. The optical scanning endoscope apparatus of claim 1, further comprising a lens configured to irradiate the illumination light emitted from the illumination fiber towards the object, wherein a reflector is provided at an outer peripheral portion of the lens, and the light amount detector for white balance detects at least a portion of the illumination light reflected by the reflector.

3. The optical scanning endoscope apparatus of claim 1, further comprising a lens configured to irradiate the illumination light emitted from the illumination fiber towards the object, wherein the light amount detector for white balance detects at least a portion of the illumination light reflected by a surface of the lens.

4. The optical scanning endoscope apparatus of claim 1, further comprising a lens configured to irradiate the illumination light emitted from the illumination fiber towards the object, wherein the light amount detector for white balance comprises a light-receiving element disposed to detect at least a portion of the illumination light at an outer peripheral portion of the lens.

5. The optical scanning endoscope apparatus of claim 1, further comprising:
a receiving fiber configured to receive light obtained from the object by irradiation with the illumination light and to guide the light obtained from the object to the photodetector, and a cap including a reflective area disposed to reflect at least a portion of the illumination light emitted from the illumination fiber so that the portion of the illumination light enters the receiving fiber;
wherein the photodetector also serves as the light amount detector for white balance.

6. The optical scanning endoscope apparatus of claim 1, further comprising:
a lens configured to irradiate the illumination light emitted from the illumination fiber towards the object; and
an optical demultiplexer configured to branch an optical path of reflected light from an optical path of the illumination light, the reflected light being yielded by a portion of the illumination light being reflected by a surface of the lens and propagated through the illumination fiber in light source direction;
wherein the light amount detector for white balance detects the reflected light branched by the optical demultiplexer.

7. An optical scanning endoscope apparatus comprising:
an illumination fiber configured to guide illumination light including light of a plurality of different wavelengths, the illumination fiber being supported to allow a tip thereof to oscillate;
a probe with the illumination fiber disposed therein, including at least a flexible portion;
a scanner configured to drive the tip of the illumination fiber and repeatedly scan the illumination light over an object;
a photodetector configured to detect light obtained from the object by scanning of the illumination light;
a signal processor configured to generate an image based on output of the photodetector;
a fiber for optical loss measurement having a same bending loss characteristic as the illumination fiber and extending into the probe at least to the flexible portion; and
a light amount detector for white balance configured to detect a light amount of light of each of the plurality of different wavelengths guided by the fiber for optical loss measurement;
wherein based on the light amount of light of each of the plurality of different wavelengths detected by the light amount detector for white balance, the optical scanning endoscope apparatus adjusts a white balance of the image that is generated.

8. The optical scanning endoscope apparatus of claim 7, wherein the fiber for optical loss measurement is provided to extend to the flexible portion of the probe and back.

9. The optical scanning endoscope apparatus of claim 1, wherein based on the light amount of light of each wavelength detected by the light amount detector for white balance, the optical scanning endoscope apparatus adjusts the white balance by controlling light source to adjust an emission intensity of light of each of the plurality of different wavelengths.

10. The optical scanning endoscope apparatus of claim 1, wherein based on the light amount of light of each wavelength detected by the light amount detector for white balance, the optical scanning endoscope apparatus adjusts the white balance by adjusting white balance of the image generated by the signal processor.
